(19) United States
(12) Plant Patent Application Publication
(10) Pub. No.: US 2013/0212756 P1
(43) Pub. Date: Aug. 15, 2013

(54) Buddleia plant named 'Hinebud2'

(50) ;

(71) Applicant: Peter C. Podaras, Davis, CA (US)

(72) Inventor: Peter C. Podaras, Davis, CA (US)

(73) Assignee: Hines Growers LLC, Laguna Hills, CA (US)

(21) Appl. No.: 13/385,241

(22) Filed: Feb. 9, 2012

(51) Int. Cl. A01H 5/00

(52) U.S. Cl. PLT/242

(57) ABSTRACT

A Buddleia plant particularly distinguished by an upright and dense growth habit, large, inflorescences that are upright-curved with a rounded tip, and is a plant suitable for use in hanging baskets, containers, and beddings, is disclosed.

### GENUS AND SPECIES

Buddleia hybrid (complex hybrid of Buddleia davidii×Buddleia alternifolia×Buddleia caryopterifolia×Buddleia lindleyana×Buddleia crispa)

### VARIETY DENOMINATION

‘Hinebud2’

### BACKGROUND OF THE NEW PLANT

The present invention comprises a new and distinct variety of Buddleia, botanically known as Buddleia hybrid, and hereinafter referred to by the variety name ‘Hinebud2’. The new variety originated from a cross-pollination conducted in Winters, Calif. in July 2010 between the female Buddleia parent, an un-named seedling (unpatented) and the male Buddleia parent, (Buddleia davidii×B. alternifolia)×B. davidii ‘Podaras #12’ (U.S. Plant Pat. No. 22,098). The female parent is a complex hybrid of Buddleia davidii×Buddleia alternifolia×Buddleia caryopterifolia×Buddleia lindleyana×Buddleia crispa. A single plant selection was subsequently chosen for further evaluation and asexual propagation.

The new plant was first asexually propagated on Sep. 15, 2010 in Winters, Calif. and has been asexually reproduced by cuttings for three generations in Winters, Calif. The present invention has been found to retain its distinctive characteristics through successive asexual propagations by softwood cuttings.

‘Hinebud2’ has not been made publicly available or sold more than one year prior to the filing of this application.

### SUMMARY OF THE INVENTION

The following are the most outstanding and distinguishing characteristics of the new variety when grown under normal horticultural practices in Winters, Calif.

1. An upright and dense growth habit;

2. Large, inflorescences that are upright-curved with a rounded tip; and

3. For use in hanging baskets, containers and beddings.

### DESCRIPTION OF THE PHOTOGRAPHS

This new Buddleia plant is illustrated by the accompanying photographs which show the overall plant habit, foliage and inflorescence of the plant. The colors are as true as can be reasonably obtained by conventional photographic procedures. The photographs are of a one-year old plant grown in a greenhouse and outdoors in Winters, Calif. in August 2011.

FIG. 1 shows a the whole plant, including the habit, foliage and inflorescence.

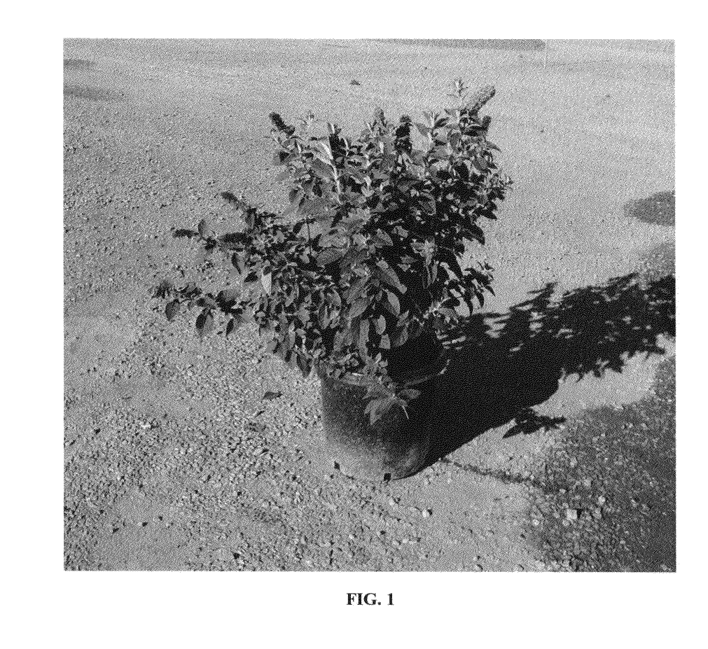

FIG. 2 shows a close-up of the inflorescence and buds.

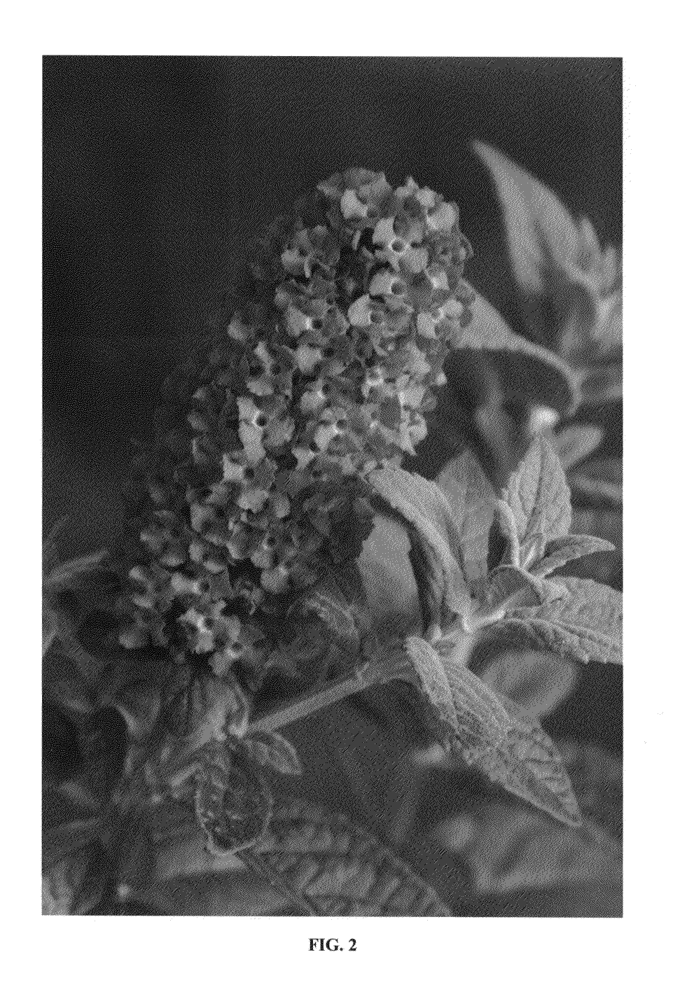

### DESCRIPTION OF THE NEW VARIETY

The following detailed descriptions set forth the distinctive characteristics of ‘Hinebud2’. The data which defines these characteristics were collected from asexual reproductions carried out in Winters, Calif. The plant history was taken in January 2012 on one and one-half year old plants grown in a greenhouse and outdoors in Winters, Calif. Color references were taken under natural light and are to the R.H.S. Colour Chart of The Royal Horticultural Society of London (R.H.S.), fifth edition (2007). Texture descriptions were taken using a dissecting microscope.

### DESCRIPTION OF THE NEW PLANT

Classification:
   Family.—Scrophulariaceae.
   Botanical name.—Buddleia hybrid (complex hybrid of Buddleia davidii×Buddleia alternifolia×Buddleia caryopterifolia×Buddleia lindleyana×Buddleia crispa).
   Common name.—Butterfly bush.
   Denomination.—‘Hinebud2’.
Plant:
   Form.—Bush.
   Habit.—Upright and dense.
   Height (measured from the top of the soil, with the plant cut back after 1 year).—183.0 cm.
   Width (horizontal plant diameter).—153.0 cm.
   Time to produce a finished plant.—3 months from a softwood cutting.
   Outdoor plant performance.—Excellent and prefers full sun.
   Time to initiate and develop roots.—About 12 days using rooting hormones between 3,000 to 6,000 ppm.
   Root description and habit.—White, fibrous, dense and branching.
   Propagation type.—Softwood cuttings.
Lateral branches:
   Quantity per plant.—Approximately 30 to 40.
   Length.—About 60.0 cm to 90.0 cm.
   Internode length.—6.0 cm to 7.0 cm.
   Diameter (from midpoint).—0.8 cm.
   Color.—RHS 144A for mature stems.
   Strength.—Strong and flexible.
   Texture.—Slightly pubescent.
   Anthocyanin.—RHS 176B.
Leaves:
   Quantity of leaves per branch.—12 to 14.
   Arrangement.—Opposite, alternating 90° after each node.
   Length.—6.0 cm to 12.0 cm.
   Width.—4.0 cm to 6.0 cm.
   Shape.—Broadly lanceolate.
   Apex.—Broadly acute.
   Base.—Attenuate.
   Margin.—Crenate.
   Color, immature.—Upper surface: RHS 138B Lower surface: RHS 148D.
   Color, mature.—Upper surface: RHS 139A Lower surface: RHS 148B.
   Texture.—Upper surface: Colliculate with stellate hairs and glandular bodies Lower surface: Extremely dense matted stellate hairs with glandular bodies.
   Venation type.—Reticulate.
   Venation color.—Upper surface: RHS N144D Lower surface: RHS 139D.
   Petiole.—Color: RHS 147C Length: 0.1 cm Diameter: 0.25 cm Strength: Flexible Texture: Pubescent.
Inflorescence:
   Height.—7.0 cm to 12.0 cm.
   Width.—4.0 cm to 6.0 cm.
   Bud.—Color: RHS 86A Shape: Conical Height: 1.0 cm Diameter: 0.4 cm.
   Flower.—Type and habit: Single and salverform shaped flowers arranged in compound terminal panicles; flowers face upright and outward; panicles are roughly conical with a slight outward curvature and a rounded apex Fragrance: Sweetly scented Flowering season: May to September Lastingness of inflorescence on the plant: About 7 to 12 days Depth: 1.0 cm Diameter: 1.2 cm Quantity per inflorescence: 300 to 600 Petals: Arrangement and quantity per flower: Four arranged in a single whorl but occasionally, there are 5 petals on flowers on the distal portion of the inflorescence; the petals fused into a narrow tube Length: 0.5 cm Width: 0.6 cm Shape: Reniform Apex: Truncate and obtuse, sometimes emarginate Base: Fused Margin: Crenate Texture: Upper surface: Colliculate Lower surface: Colliculate with sparse stellate trichomes Color, when opening: Upper surface: RHS 86B Lower surface: RHS N82B Color, when open: Upper surface: RHS N87A Lower surface: RHS N87B Tube: Length: 0.9 cm Width: 0.2 cm Color: Inside: RHS N34A Outside: RHS N92A Texture: Inside: Colliculate with long, upward-setose pointed hairs Outside: Colliculate with stellate hairs Calyx: Arrangement and quantity: Four sepals (occasionally 5 on flowers on the distal end of the inflorescence) arranged in a single whorl and fused halfway up from the base Shape: Tubular, fitting snugly over the corolla tube Sepals: Length: 0.4 cm Width: 0.1 cm Shape: Lanceolate but fused halfway up from the base Apex: Broadly acute Base: Fused with other sepals Margin: Slightly sinuate Texture: Upper surface: Lepidote with stellate hairs Lower surface: Colliculate with more sparse stellate hairs than the upper surface Color: Upper surface: RHS 147C Lower surface: RHS 146B Peduncle: Length: 5.0 cm to 7.5 cm Diameter: 0.2 cm Strength: Flexible Color: RHS 142D Texture: Pubescent Pedicel: Length: 0.2 cm to 0.4 cm Diameter: 0.15 cm Color: RHS 139D Texture: Pubescent.
Reproductive organs:
   Stamens.—Quantity per flower: Mostly 4, occasionally 5 Filament: Completely fused to inside of corolla tube; cannot determine length or color Anther: Length: 0.12 cm Color: RHS 197D Pollen amount: Sparse Pollen color: RHS 197D.
   Pistil.—Quantity per flower: 1 Length: 0.15 cm Stigma: Shape: Club-shaped, slight bilateral cleavage at the distal end Color: RHS 137A Style: Length: 0.1 cm Color: RHS 192A Ovary: Color: RHS 192A.
Fruit and seed set: Sparse seed set. Seeds are RHS 158C, the shape is long and narrow, about 0.025 cm by 0.01 cm.
Disease and insect resistance: Susceptible to spider mites

### Comparison with Parental and Commercial Variety

‘Hinebud2’ differs from the un-named female Buddleia parent (unpatented) in that ‘Hinebud2’ has larger leaves and an upright habit, while the female parent has smaller leaves and a prostrate habit.

‘Hinebud2’ differs from the male Buddleia parent ‘Podaras #12’ (U.S. Plant Pat. No. 22,098) in that ‘Hinebud2’ is a taller and has a greater horizontal plant diameter (height is 183.0 cm and width or horizontal plant diameter is 153.0 cm) than ‘Podaras #12’ (height is approximately 23.0 cm and width is 38.0 cm to 56.0 cm).

‘Hinebud2’ differs from the commercial comparison ‘Blue Chip’ (U.S. Plant Pat. No. 19,991) in that ‘Hinebud2’ has longer internodes (6.0 cm to 7.0 cm) and longer peduncles (5.0 cm to 7.5 cm) than ‘Blue Chip’ (internode is 3.6 cm in length and peduncles are 2.4 cm in length).

I claim:

1. A new and distinct variety of Buddleia plant as shown and described herein.
